(19) United States
(12) Patent Application Publication
(10) Pub. No.: US 2021/0386720 A1
(43) Pub. Date: Dec. 16, 2021

(54) METHODS OF TREATING CNS TUMORS WITH TESETAXEL

(71) Applicant: Odonate Therapeutics, Inc., San Diego, CA (US)

(72) Inventors: Joyce James, Oakdale, CA (US); Kevin Tang, La Jolla, CA (US); Stew Kroll, Oakland, CA (US); John G. Lemkey, Rancho Santa Fe, CA (US); Steven Pfeiffer, Camarillo, CA (US); Jeff Vacirca, Shoreham, NY (US); Thomas Wei, New York, NY (US)

(21) Appl. No.: 17/286,577

(22) PCT Filed: Sep. 5, 2019
(86) PCT No.: PCT/US19/49642

§ 371 Date: Apr. 19, 2021

Related U.S. Application Data
(60) Provisional application No. 62/811,181, filed on Feb. 27, 2019.
(60) Provisional application No. 62/746,926, filed on Oct. 17, 2018.

(51) Int. Cl. A61K 31/443; A61K 31/7068; A61K 39/395; A61P 35/00

(52) U.S. Cl. CPC A61K 31/443; A61P 35/00; A61K 39/3955; A61K 31/7068

(57) ABSTRACT

The present disclosure provides methods for treating a patient with a cancer in the central nervous system, such as a cancer that is a metastasis of a primary cancer, comprising administering tesetaxel and capecitabine to the patient.

### CROSS REFERENCE TO RELATED APPLICATIONS

This patent application claims the benefit of priority to U.S. Provisional Patent Application No. 62/746,926, filed Oct. 17, 2018, and U.S. Provisional Patent Application No. 62/811,181, filed Feb. 27, 2019, each of which is incorporated by reference herein in its entirety.

### BACKGROUND OF THE INVENTION

The 5-year survival rate for people with cancerous central nervous system (CNS) tumors is 34% for men and 36% for women. CNS tumors frequently occur by metastasis of other cancers, elsewhere in the body, that spread or metastasize to the brain. In adults, secondary (metastatic) CNS tumors are far more common than primary CNS tumors (those originating in the CNS itself, or tissues close to it). While any cancer can spread to the brain, the two most common types to do so are breast cancer and lung cancer.

Breast cancer is the most common cancer in women worldwide, with an estimated 2.1 million new cases diagnosed per year. In Europe, an estimated 523,000 new cases are diagnosed and approximately 138,000 women will die of the disease each year, making it the leading cause of cancer death in women. In the United States (U.S.), an estimated 269,000 new cases are diagnosed and approximately 41,000 women will die of the disease each year, making it the second-leading cause of cancer death in women.

Breast cancer is a heterogeneous disease comprised of several molecular subtypes, which are commonly grouped into clinical subtypes based on receptor status. Receptors that are assessed in standard clinical practice include the estrogen receptor (ER) and the progesterone receptor (PR), which are collectively referred to as the hormone receptors (HR), and human epidermal growth factor receptor 2 (HER2). Breast cancers generally are categorized by the presence or absence of these receptors. The most common form of breast cancer is HER2 negative and HR positive, accounting for approximately 64% of newly diagnosed cases. HER2 positive breast cancer and triple negative breast cancer (TNBC), which lacks all three receptors, are less common, accounting for approximately 13% and 11% of breast cancers, respectively.

Breast cancer typically is staged (Stage 0-IV) based on the size of the tumor, whether or not the tumor is invasive, whether or not the cancer is in the lymph nodes and whether or not the cancer has spread (metastasized) to other parts of the body beyond the breast, most often the bones, lungs, liver, or brain. The prognosis for women with locally advanced or metastatic breast cancer (LA/MBC) remains poor; the 5-year survival rate for metastatic disease is about 22%, making this an area of continued, high unmet medical need. The existence and/or development of CNS metastases typically worsens outcomes in patients with cancer, including LA/MBC. New methods for treating CNS metastases are needed.

### SUMMARY OF THE INVENTION

Tesetaxel is a novel, highly potent, orally administered taxane. Taxanes are an established class of anticancer agents that are broadly used in various cancers, including breast cancer. The primary pharmacologic mechanism of tesetaxel, like other taxanes, is to stabilize cellular microtubule formation (inhibit tubulin depolymerization) in rapidly dividing cells, leading to arrest of unscheduled cell division at the G₂/M phase of the cell cycle and cell death. Tesetaxel has several pharmacologic properties that make it unique among taxanes:
Tesetaxel is a capsule for oral administration with a low pill burden;
Tesetaxel has a long (˜8-day) terminal plasma half-life (t_{1/2}) in humans, enabling the maintenance of adequate drug levels with relatively infrequent dosing;
Tesetaxel's formulation does not contain polyoxyethylated castor oil or polysorbate 80, solubilizing agents contained in other taxane formulations known to cause hypersensitivity reactions; and
Tesetaxel has been shown to retain activity against taxane-resistant tumors in nonclinical studies.

Tesetaxel retains the same taxane core as the approved taxanes, but includes the addition of two novel, nitrogen-containing functional groups. Tesetaxel is chemically designed to: (1) not be substantially effluxed by the P-glycoprotein (P-gp) pump, with the intent of retaining activity against chemotherapy-resistant tumor cells; (2) have high oral bioavailability; (3) have high solubility; and (4) have a long t_{1/2} in humans.

In some aspects, the present disclosure provides a method of treating a cancer in the CNS of a human patient, comprising administering a therapeutically-effective amount of tesetaxel systemically (for example, on day 1 of a 21-day cycle). In certain embodiments, the method further comprises administering a therapeutically effective amount of capecitabine daily (preferably divided into two daily doses) starting on day 1 of the 21-day cycle for 14 consecutive 24-hour periods.

### DETAILED DESCRIPTION OF THE INVENTION

It has been discovered that tesetaxel is brain-penetrant; that is, it crosses the blood-brain barrier. This result is unexpected because other taxanes, such as docetaxel and paclitaxel, have not been found to be effective against CNS metastases. Accordingly, tesetaxel, unlike docetaxel and paclitaxel, may be conveniently utilized in the treatment of tumors of the CNS, such as brain tumors. The structures of tesetaxel, docetaxel and paclitaxel are shown below:

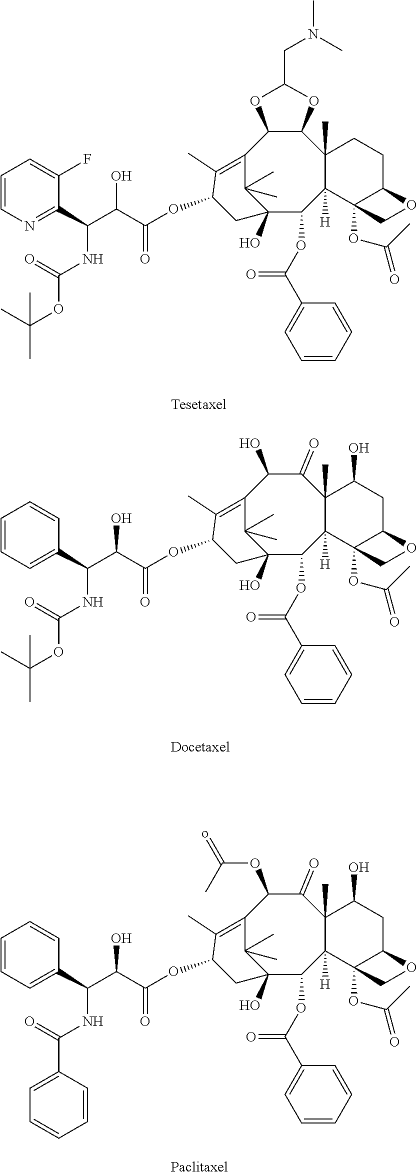

In addition, tesetaxel and capecitabine may be effectively used in conjoint therapy, as described in International Patent Application PCT/US18/35653, which is hereby incorporated by reference herein in its entirety. When so used, the combination can provide greater efficacy than capecitabine alone. For instance, the methods disclosed herein may result in longer progression-free survival, longer survival, a greater treatment response, a longer duration of response and/or better disease control. In some embodiments, the combination is at least as efficacious as administration of capecitabine alone (e.g., at a dose of 2,500 mg/m² or 2,000 mg/m² daily for 14 consecutive days of a 21-day cycle), but with a more tolerable safety profile. More tolerable treatment regimens, such as those disclosed herein, are more likely to be continued by patients, and thus may be more likely to be effective.

In some aspects, the present disclosure provides a method of treating a cancer in the CNS of a human patient, comprising administering a therapeutically effective amount of tesetaxel systemically. In some embodiments, the tesetaxel is administered orally.

In some embodiments, the cancer comprises a metastatic tumor, that is, the CNS cancer arises from a primary cancer located elsewhere in the patient's body. In some embodiments, the metastatic tumor is a metastasis of a primary cancer selected from breast cancer or lung cancer.

In some embodiments, the metastatic tumor is a metastasis of a primary breast cancer. In some embodiments, the breast cancer is HR positive. In some embodiments, the patient has previously received endocrine therapy. In some embodiments, the breast cancer is ER positive. In some embodiments, the breast cancer is PR positive. In some embodiments, the breast cancer is HER2-negative. In some embodiments, the breast cancer is HR positive and HER2-negative. In some embodiments, the breast cancer is HR negative and HER2-negative. In some embodiments, the breast cancer is HER2-positive.

In some embodiments, the metastatic tumor is a metastasis of a primary lung cancer, such as non-small cell lung cancer (NSCLC) or small cell lung cancer. In some embodiments, the lung cancer is NSCLC. In some embodiments, the lung cancer is small cell lung cancer. In some embodiments, the lung cancer is of squamous histology. In some embodiments, the lung cancer is of non-squamous histology.

In some embodiments, the cancer comprises a primary CNS tumor. In some embodiments, the primary CNS tumor is an acoustic neuroma, astrocytoma, chordoma, CNS lymphoma, craniopharyngioma, glioma, medulloblastoma, meningioma, oligodendroglioma, pituitary tumor, primitive neuroectodermal or schwannoma.

The tesetaxel may be administered at any suitable dosage and on any suitable schedule. In some embodiments, the method comprises administering the tesetaxel on day 1 of a 21-day cycle. In some embodiments, administering a therapeutically effective amount of tesetaxel comprises administering 18-31 mg/m² of tesetaxel on day 1 of the 21-day cycle. In some embodiments, administering a therapeutically effective amount of tesetaxel comprises administering 27 mg/m² of tesetaxel on day 1 of the 21-day cycle.

The treatment cycle may be repeated as necessary. In some embodiments, the method comprises repeating the 21-day cycle at least once. In some embodiments, the method comprises repeating the 21-day cycle until the cancer progresses or until unacceptable toxicity is observed.

The tesetaxel may also be conjointly administered with other suitable therapeutic agents, such as capecitabine. In some embodiments, the method comprises administering a therapeutically effective amount of tesetaxel and a therapeutically effective amount of capecitabine conjointly. In some such embodiments, when the tesetaxel is administered on day 1 of a 21-day cycle, the method further comprises administering capecitabine daily, starting on day 1 of the 21-day cycle for 14 consecutive 24-hour periods.

Any suitable dose of capecitabine may be used. When a daily dosage of capecitabine is specified, the daily dosage may be divided into a number of smaller, divided doses, such as two, three, four, five, six, or more divided doses. In some preferred embodiments, the daily dosage of capecitabine is divided into two divided doses. When administering divided doses, a daily dosage regimen may begin with a partial dose on the first day, and end with a partial dose on the last day, such that the daily dosage is delivered in a number of 24-hour periods, which may or may not correspond to calendar days. Thus, dosing of capecitabine is alternately discussed herein in terms of the total daily dosage (i.e., the total amount administered in a day or in a 24-hour period) or in terms of divided doses (i.e., the individual doses administered over the course of a day or a 24-hour period that combine to meet the total daily dosage).

In some embodiments, capecitabine is administered at twice-daily intervals (i.e., two times per 24-hour period) for a period of time, such as for 14 consecutive 24-hour periods. In some such embodiments, which are further described below, a first dose of capecitabine is administered on day 1, and subsequent doses are administered at twice-daily intervals with a final dose administered on day 15. In other such embodiments, which are further described below, capecitabine is administered twice daily for 14 consecutive calendar days (i.e., two doses of capecitabine are administered on each of days 1-14). Thus, reference to a number of “daily dosages” of capecitabine herein refers to administering capecitabine for that number of 24-hour periods and encompasses administering capecitabine for that number of calendar days.

In some embodiments, administering a therapeutically effective amount of capecitabine comprises administering capecitabine twice daily on days 1-14 of the 21-day cycle. In some embodiments, the method comprises administering a therapeutically effective amount of capecitabine in 28 doses at twice-daily intervals beginning on day 1 of the 21-day cycle. In some embodiments, the method comprises administering a first dose of capecitabine on day 1 of the 21-day cycle and administering a final 28^{th} dose on day 15 of the 21-day cycle. In some such embodiments, administering a therapeutically effective amount of capecitabine comprises administering a first dose of capecitabine after noon (e.g., in the evening) of day 1 of the 21-day cycle and administering a final 28^{th} dose before noon (e.g., in the morning) on day 15 of the 21-day cycle.

In some embodiments, administering a therapeutically effective amount of capecitabine comprises administering 14 daily dosages of 300-2,000 mg/m² (such as 1,000-1,800 mg/m²) of capecitabine beginning on day 1 of the 21-day cycle. In some embodiments, administering a therapeutically effective amount of capecitabine comprises administering 14 daily dosages of 1,650 mg/m² of capecitabine, beginning on day 1 of the 21-day cycle.

In some embodiments, administering a therapeutically effective amount of capecitabine comprises administering 825 mg/m² of capecitabine at twice-daily intervals for 14 consecutive 24-hour periods beginning on day 1 of the 21-day cycle. In some such embodiments, administering a therapeutically effective amount of capecitabine comprises administering 825 mg/m² of capecitabine twice daily on days 1-14 of the 21-day cycle. In other such embodiments, administering capecitabine comprises administering a first dose of 825 mg/m² of capecitabine on day 1, administering subsequent doses of 825 mg/m² of capecitabine at twice-daily intervals, and administering a final dose of 825 mg/m² of capecitabine on day 15. In some embodiments, administering a therapeutically effective amount of capecitabine comprises administering 825 mg/m² of capecitabine at twice-daily intervals for 14 consecutive 24-hour periods beginning on day 1 of the 21-day cycle.

In some embodiments, administering a therapeutically effective amount of capecitabine comprises administering 14 daily dosages of 1,750 mg/m² of capecitabine beginning on day 1 of the 21-day cycle. In some embodiments, administering a therapeutically effective amount of capecitabine comprises administering 875 mg/m² of capecitabine at twice-daily intervals for 14 consecutive 24-hour periods beginning on day 1 of the 21-day cycle. In some such embodiments, administering a therapeutically effective amount of capecitabine comprises administering 875 mg/m² twice daily on days 1-14 of the 21-day cycle. In other such embodiments, administering a therapeutically effective amount of capecitabine comprises administering a first dose of 875 mg/m² on day 1, administering subsequent doses of 875 mg/m² of capecitabine at twice-daily intervals, and administering a final dose of 875 mg/m² of capecitabine on day 15.

In some embodiments, administering a therapeutically effective amount of capecitabine comprises administering 28 doses of 150-1,000 mg/m² of capecitabine at twice-daily intervals. In some embodiments, administering a therapeutically effective amount of capecitabine comprises administering 150-1,000 mg/m² of capecitabine at twice-daily intervals for 14 consecutive 24-hour periods. In some such embodiments, administering a therapeutically effective amount of capecitabine comprises administering 150-1,000 mg/m² twice daily on days 1-14 of the 21-day cycle. In other such embodiments, administering a therapeutically effective amount of capecitabine comprises administering a first dose of 150-1,000 mg/m² of capecitabine on day 1, and administering subsequent doses of 150-1,000 mg/m² of capecitabine at twice-daily intervals and concluding by administering a final dose of 150-1,000 mg/m² of capecitabine on day 15.

In some embodiments, administering a therapeutically effective amount of capecitabine comprises administering 28 doses of 150-1,000 mg/m² of capecitabine at twice-daily intervals beginning with the first dose on day 1 of the 21-day cycle and ending with the 28^{th} dose on day 15 of the 21-day cycle. In some embodiments, administering a therapeutically effective amount of capecitabine comprises administering 28 doses of 825 mg/m² of capecitabine at twice-daily intervals. In some embodiments, administering a therapeutically effective amount of capecitabine comprises administering 28 doses of 825 mg/m² of capecitabine at twice-daily intervals beginning with the first dose on day 1 of the 21-day cycle and ending with the 28^{th} dose on day 15 of the 21-day cycle. In some embodiments, administering a therapeutically effective amount of capecitabine comprises administering 28 doses of 875 mg/m² of capecitabine at twice-daily intervals. In some embodiments, administering a therapeutically effective amount of capecitabine comprises administering 28 doses of 875 mg/m² of capecitabine at twice-daily intervals beginning with the first dose on day 1 of the 21-day cycle and ending with the 28^{th} dose on day 15 of the 21-day cycle.

In some embodiments, the patient has previously been treated with a taxane. In some embodiments, the patient has previously been treated with a taxane in the neoadjuvant or adjuvant setting. In some embodiments, the taxane is paclitaxel, docetaxel or albumin-bound (nab) paclitaxel. In some embodiments, the patient has not previously been treated with a taxane.

In some aspects, the present disclosure provides a method of treating a cancer in the CNS of a human patient comprising: administering tesetaxel (e.g., 18-31 mg/m² of tesetaxel) on day 1 of a 21-day cycle; and administering 28 doses of capecitabine (e.g., 825 mg/m² of capecitabine) at twice-daily intervals beginning on day 1 of the 21-day cycle. In some embodiments, 27 mg/m² of tesetaxel is administered on day 1 of the 21-day cycle. In some embodiments, each dose of capecitabine administered at a twice-daily interval is 875 mg/m². In some embodiments, each dose of capecitabine administered at a twice-daily interval is 150-1,000 mg/m². In some such embodiments, each dose of capecitabine administered at a twice-daily interval is 300-1,000 mg/m², 450-1,000 mg/m², 600-1,000 mg/m², 750-1,000 mg/m², or 750-900 mg/m².

In some aspects, the present disclosure provides a method of treating a cancer in the CNS of a human patient comprising: administering tesetaxel (e.g., 18-31 mg/m² of tesetaxel) on day 1 of a 21-day cycle; and administering capecitabine (e.g., 1,650 mg/m² of capecitabine) daily on days 1-14 of the 21-day cycle. In some embodiments, 27 mg/m² of the tesetaxel is administered on day 1 of the 21-day cycle. In some embodiments, 1,750 mg/m² of capecitabine is administered on days 1-14 of the 21-day cycle. In some embodiments, 300-2,000 mg/m² of capecitabine is administered on days 1-14 of the 21-day cycle. In some such embodiments, 600-2,000 mg/m², 900-2,000 mg/m², 1,200-2,000 mg/m², 1,500-2,000 mg/m² or 1,500-1,800 mg/m² of capecitabine is administered on days 1-14 of the 21-day cycle.

In some aspects, the present disclosure provides a method of treating a cancer in the CNS of a human patient comprising: administering tesetaxel (e.g., 18-31 mg/m² of tesetaxel) on day 1 of a 21-day cycle; and administering capecitabine (e.g., 825 mg/m² of capecitabine) at twice-daily intervals beginning with the first dose on day 1 of the 21-day cycle (e.g., in the evening) and ending with the 28^{th} dose on day 15 of the 21-day cycle (e.g., in the morning). In some embodiments, 27 mg/m² of tesetaxel is administered on day 1 of the 21-day cycle. In some embodiments, 825 mg/m² of capecitabine is administered at twice-daily intervals beginning with the first dose on day 1 of the 21-day cycle and ending with the 28^{th} dose on day 15 of the 21-day cycle. In some embodiments, 875 mg/m² of capecitabine is administered at twice-daily intervals beginning with the first dose on day 1 of the 21-day cycle and ending with the 28^{th} dose on day 15 of the 21-day cycle. In some embodiments, 150-1,000 mg/m² of capecitabine is administered at twice-daily intervals beginning with the first dose on day 1 of the 21-day cycle and ending with the 28^{th} dose on day 15 of the 21-day cycle. In some such embodiments, 300-1,000 mg/m², 450-1,000 mg/m², 600-1,000 mg/m², 750-1,000 mg/m² or 750-900 mg/m² of capecitabine is administered at twice-daily intervals beginning with the first dose on day 1 of the 21-day cycle and ending with the 28^{th} dose on day 15 of the 21-day cycle.

In preferred embodiments, the daily dosage of capecitabine is divided into two doses on the days in which it is administered. Thus, in some embodiments, administering capecitabine comprises administering capecitabine twice daily on days 1-14 of the 21-day cycle (e.g., administering capecitabine 825 mg/m² twice daily on days 1-14 of the 21-day cycle or administering capecitabine 875 mg/m² twice daily on days 1-14 of the 21-day cycle). In certain embodiments, a regimen of twice-daily dosing (e.g., twice in a calendar day), or dosing at twice-daily intervals (e.g., twice in a 24-hour period), may begin or end in the middle of a calendar day, such that only one dose is administered on the first calendar day of the regimen and/or the last calendar day of the regimen. In certain embodiments where twice-daily dosing, or dosing at twice-daily intervals, is used, only one dose is administered on the first calendar day of dosing, (e.g. in the evening). In certain such embodiments, only one dose is administered on the last calendar day of dosing, which, for a 28-dose regimen, would be the 15^{th} calendar day of the cycle (e.g., in the morning).

In some embodiments, the 21-day cycle is repeated one or more times, such that the 21-day cycle is administered 2, 3, 4, 5 or more times. According to these embodiments, within each iteration of the 21-day cycle, tesetaxel is administered on day 1 and capecitabine is administered on days 1-14, as described herein. Alternatively, within each iteration of the 21-day cycle, tesetaxel may be administered on day 1 and capecitabine may be administered as 28 doses of capecitabine (e.g., 825 mg/m² of capecitabine) at twice-daily intervals beginning on day 1 of the 21-day cycle. In some embodiments, the 21-day cycle is repeated until the cancer progresses or until unacceptable toxicity is observed.

In some embodiments, the method further comprises administering a therapeutically effective amount of an inhibitor of programmed cell death protein 1 (PD-1) or programmed death-ligand 1 (PD-L1), such as nivolumab, pembrolizumab or atezolizumab. In some such embodiments, the inhibitor of PD-1 or PD-L1 is administered on day 1 of the 21-day cycle. In some such embodiments, the inhibitor is administered by intravenous infusion. In some such embodiments, the intravenous infusion occurs over 30 minutes. In other such embodiments, the intravenous infusion occurs over 60 minutes.

Any suitable dose of the inhibitor of PD-1 or PD-L1 may be used. In some embodiments, 360 mg of nivolumab is administered, such as by intravenous infusion, such as over 30 minutes. In some embodiments, 200 mg of pembrolizumab is administered, such as by intravenous infusion, such as over 30 minutes. In some embodiments, 1,200 mg of atezolizumab is administered, such as by intravenous infusion, such as over 30 minutes or over 60 minutes. In some such embodiments, the first infusion of atezolizumab is administered over 60 minutes and, if it is tolerated, all subsequent infusions (e.g., subsequent infusions of atezolizumab on day 1 of subsequent 21-day cycles) are delivered over 30 minutes.

In some embodiments, the conjoint therapy described herein is administered to a patient who has previously been treated with a taxane (e.g., paclitaxel, docetaxel or nab-paclitaxel). In certain preferred embodiments, the conjoint therapy described herein is administered to a patient who has previously been treated with a taxane in the neoadjuvant or adjuvant setting. In certain embodiments, the patient's cancer is taxane-resistant (e.g., the cancer is resistant to treatment with at least one taxane). In certain embodiments, the cancer has relapsed less than six months after the discontinuation of the prior taxane therapy. In certain embodiments, the cancer has relapsed six to twelve months after the discontinuation of the prior taxane therapy. In certain embodiments, the cancer has relapsed twelve months or more after the discontinuation of the prior taxane therapy.

In some embodiments, the primary cancer is breast cancer, such as MBC or LA/MBC. In some embodiments, the breast cancer is locally advanced breast cancer. In some embodiments, the breast cancer is HR positive, such as ER positive or PR positive. In some embodiments, the patient has previously received endocrine therapy. In some embodiments, the breast cancer is HER2-negative. In some embodiments, the breast cancer is HR positive and HER2-negative. In some embodiments, the breast cancer is HER2-positive. In some embodiments, the breast cancer is HR-negative (i.e., ER negative and PR negative) and HER2-negative.

### Definitions

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population (e.g., by a statistically and/or clinically significant amount).

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal), then the treatment is prophylactic (i.e., it protects the host against developing the unwanted condition); whereas, if it is administered after manifestation of the unwanted condition, the treatment is therapeutic (i.e., it is intended to diminish, ameliorate or stabilize the existing unwanted condition or side effects thereof).

The phrase “therapeutically effective amount” means the concentration of a compound that is sufficient to elicit the desired therapeutic effect.

The phrases “conjoint administration” and “administered conjointly” refer to any form of administration of two or more different therapeutic compounds such that the second compound is administered while the previously administered therapeutic compound is still therapeutically effective in the body (e.g., the two compounds are simultaneously therapeutically effective in the patient, which may include synergistic effects of the two compounds). For example, the different therapeutic compounds can be administered either in the same formulation or in a separate formulation, either concomitantly (i.e., at substantially the same time) or sequentially (i.e., with one compound administered first and the other compound administered at a later time). In certain embodiments, the different therapeutic compounds can be administered within one hour, 12 hours, 24 hours, 36 hours, 48 hours, 72 hours, 7 days, 14 days or 15 days of one another, or wherein the different therapeutic compounds are administered within the same treatment cycle as one another. Thus, an individual who receives such treatment can benefit from a combined effect of different therapeutic compounds.

The term “prodrug” is intended to encompass compounds which, under physiologic conditions, are converted into the therapeutically active agents of the present invention. A common method for making a prodrug is to include one or more selected moieties that are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of the invention in a formulation represented above can be replaced with the corresponding suitable prodrug (e.g., wherein a hydroxyl in the parent compound is presented as an ester or a carbonate or carboxylic acid present in the parent compound is presented as an ester).

Tesetaxel is a taxane having the following structure:

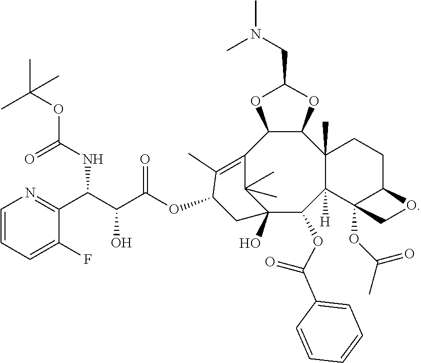

Tesetaxel and its preparation are described in U.S. Pat. No. 6,677,456, which is incorporated by reference in its entirety. Various crystal forms of tesetaxel are described in U.S. Pat. No. 7,410,980, which is hereby incorporated by reference in its entirety.

### Pharmaceutical Compositions

The compositions and methods of the present invention may be utilized to treat an individual in need thereof. In certain embodiments, the individual is a human. When administered, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils, such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as a tablet, capsule (including sprinkle capsule and gelatin capsule), granule, lyophile for reconstitution, powder, solution, syrup, suppository, injection or the like. The composition can also be present in a transdermal delivery system (e.g., a skin patch). The composition can also be present in a solution suitable for topical administration, such as an eye drop.

A pharmaceutically acceptable carrier can contain physiologically acceptable agents that act, for example, to stabilize, to increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response or other problems or complications, commensurate with a reasonable benefit-risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials that can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (e.g., as drenches in aqueous or non-aqueous solutions or suspensions, tablets, capsules [including sprinkle capsules and gelatin capsules], boluses, powders, granules or pastes for application to the tongue); absorption through the oral mucosa (e.g., sublingually); anally, rectally or vaginally (e.g., as a pessary, cream or foam); parenterally (including intramuscularly, intravenously, subcutaneously or intrathecally as, for example, a sterile solution or suspension); nasally; intraperitoneally; subcutaneously; transdermally (e.g., as a patch applied to the skin); and topically (e.g., as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient that can be combined with a carrier material to produce a single dosage form will vary depending upon the host being treated and the particular mode of administration. The amount of active ingredient that can be combined with a carrier material to produce a single dosage form will generally be that amount of the compound that produces a therapeutic effect. Generally, out of 100 percent, this amount will range from about 1 percent to about 99 percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules [including sprinkle capsules and gelatin capsules], tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high-molecular-weight polyethylene glycols and the like.

A tablet may be made by compression or molding, optionally with one or more accessory ingredients. Compressed tablets may be prepared using a binder (e.g., gelatin or hydroxypropylmethyl cellulose), lubricant, inert diluent, preservative, disintegrant (e.g., sodium starch glycolate or cross-linked sodium carboxymethyl cellulose) or surface-active or dispersing agent. Molded tablets may be made by molding, in a suitable machine, a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain pacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature but liquid at body temperature and, therefore, will melt in the rectum or vaginal cavity and release the active compound.

Formulations of the pharmaceutical compositions for administration to the mouth may be presented as a mouthwash, oral spray or oral ointment.

Alternatively or additionally, compositions can be formulated for delivery via a catheter, stent, wire or other intraluminal device. Delivery via such devices may be especially useful for delivery to the bladder, urethra, ureter, rectum or intestine.

Formulations that are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to an active compound, excipients such as lactose, talc, silicic acid, aluminum hydroxide, calcium silicates and polyamide powder or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like are also contemplated as being within the scope of this invention. Exemplary ophthalmic formulations are described in U.S. Publication Nos. 2005/0080056, 2005/0059744, 2005/0031697 and 2005/004074 and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids. A preferred route of administration is local administration (e.g., topical administration, such as eye drops, or administration via an implant).

The phrases “parenteral administration” and “administered parenterally” as used herein mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders, which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol and the like) and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid and the like. It may also be desirable to include isotonic agents, such as sugars, sodium chloride and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5%, more preferably, 0.5 to 90%, of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition and mode of administration, without being toxic to the patient.

The selected dosage level will depend upon a variety of factors, including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated and like factors well known in the medical arts.

In general, a suitable daily dosage of an active compound used in the compositions and methods of the invention will be that amount of the compound that is the lowest dose effective to produce a therapeutic effect. Such an effective dose will generally depend upon the factors described above.

If desired, the effective daily dosage of the active compound may be administered as one, two, three, four, five, six or more sub-doses (or divided doses) administered separately at appropriate intervals throughout a day, optionally, in unit dosage forms. In preferred embodiments of the present invention, an active compound may be administered one or two times daily on the days on which it is administered.

In certain embodiments, the methods of the invention may be used alone or the compounds administered may be used conjointly with another type of therapeutic agent.

This invention includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, L-ascorbic acid, L-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, D-glucoheptonic acid, D-gluconic acid, D-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, L-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, L-pyroglutamic acid, salicylic acid, sebacic acid, stearic acid, succinic acid, sulfuric acid, L-tartaric acid, thiocyanic acid, p-toluenesulfonic acid, trifluoroacetic acid, and undecylenic acid salts.

The pharmaceutically acceptable acid-addition salts can also exist as various solvates, such as with water, methanol, ethanol, dimethylformamide and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### EXEMPLIFICATION

The invention now being generally described will be more readily understood by reference to the following examples, which are included merely for purposes of illustration of certain aspects and embodiments of the present invention, and are not intended to limit the invention.

### Example 1

### Clinical Study

Patients with HER2 negative, HR positive LA/MBC previously treated with a taxane in the neoadjuvant or adjuvant setting are recruited and randomized into one of two treatment arms. Known metastases to the CNS are permitted but not required. An additional analysis of CNS tumor efficacy will be conducted for patients with CNS tumor metastases.

Patients in arm 1 are treated orally with 27 mg/m² of tesetaxel on Day 1 of a 21-day cycle and 14 daily dosages of 1,650 mg/m² of capecitabine (825 mg/m² at twice-daily intervals) starting on Day 1 and ending on Day 15 of a 21-day cycle, beginning with the evening dose on Day 1 and ending with the morning dose on Day 15 of each 21-day cycle. Treatment continues in 21-day cycles until the disease progresses or unacceptable toxicity is observed in the patient.

Patients in arm 2 are treated with 14 daily dosages of 2,500 mg/m² of capecitabine (1,250 mg/m² at twice-daily intervals) starting on Day 1 and ending on Day 15 of a 21-day cycle, beginning with the evening dose on Day 1 and ending with the morning dose on Day 15 of each 21-day cycle. Treatment continues in 21-day cycles until the disease progresses or unacceptable toxicity is observed in the patient.

The primary endpoint of the study is progression-free survival as adjudicated by an independent review committee. Secondary endpoints include overall survival, objective response rate and disease control rate. CNS metastases efficacy endpoints include CNS objective response rate, CNS duration of response and CNS progression-free survival as adjudicated by a CNS independent review committee and CNS overall survival.

### Example 2

### Clinical Study

Elderly patients (age≥65) with HER2 negative LA/MBC not previously treated with chemotherapy for LA/MBC are recruited into one treatment arm without randomization. Inclusion criteria include prior endocrine therapy with or without a CDK 4/6 inhibitor unless endocrine therapy is not indicated. Known CNS metastases are permitted but not required.

Patients are treated with 27 mg/m² of tesetaxel monotherapy orally once on Day 1 of each 21-day cycle. Treatment continues in 21-day cycles until documentation of progressive disease (PD), unacceptable toxicity observed in the patient or other decision(s) to discontinue treatment.

The primary endpoint of the study is objective response rate as assessed by the investigators using RECIST 1.1 criteria. Secondary endpoints include progression-free survival as assessed by the investigators using RECIST 1.1 criteria and overall survival. Efficacy for CNS metastases is measured by the CNS objective response rate and CNS duration of response.

### Example 3

### Clinical Study

Adult patients (age ≥18) with triple negative LA/MBC who have not received prior chemotherapy for LA or metastatic disease are recruited and randomized into three treatment arms. The patients' most recent biopsy must be HR negative. Known metastases to the CNS are permitted but not required.

Patients are treated with 27 mg/m² of tesetaxel orally on Day 1 of each 21-day cycle, plus one of the following: (Al) nivolumab (360 mg) by 30-minute intravenous infusion on Day 1 of each 21-day cycle; (A2) pembrolizumab (200 mg) by 30-minute intravenous infusion on Day 1 of each 21-day cycle; or (A3) atezolizumab (1,200 mg) by 60-minute intravenous infusion (if first infusion is tolerated, all subsequent infusions may be delivered over 30 minutes) on Day 1 of each 21-day cycle. Treatment continues in 21-day cycles until documentation of progressive disease, unacceptable toxicity observed in the patient, or other decision(s) to discontinue treatment.

The primary endpoint of the study is objective response rate as assessed by the investigators using RECIST 1.1 criteria. Secondary endpoints include progression-free survival as assessed by the investigators using RECIST 1.1 criteria and overall survival. Efficacy for CNS metastases is measured by the CNS objective response rate and CNS duration of response.

### Example 4

### Clinical Study

Patients with HER2 negative, HR positive LA/MBC who have not have received a taxane in the neoadjuvant, adjuvant or metastatic setting (and, where indicated, have progressed on endocrine therapy) are recruited into a single arm. Known metastases to the CNS are permitted but not required. Two cohorts are enrolled in parallel.

First Cohort

Patients in the first cohort are administered 27 mg/m² tesetaxel orally once on Day 1 of each 21-day cycle and 825 mg/m² capecitabine orally at twice-daily intervals beginning with the evening dose on Day 1 through the morning dose on Day 15 of each 21-day cycle.

Second Cohort

The second cohort is designed to collect pharmacokinetic data (“PK”) on a dense sampling schedule for tesetaxel, and to study the potential PK drug-drug interaction of tesetaxel on capecitabine and its active metabolite, 5-fluorouracil (5-FU). Patients in Cohort 2 are randomized 1:1 to receive on Cycle 1, Day -1 either a single dose of capecitabine at a reduced dose level of 825 mg/m² (Cohort 2A) or a dose level of 1,250 mg/m² (Cohort 2B). Specifically, on Day -1, following an overnight fast of at least 8 hours, patients are administered a single, morning dose of capecitabine in the clinic within 10 minutes following a standard breakfast meal. PK sample collection occurs from pre-capecitabine dose through 4 hours post-capecitabine dose. Patients do not receive the evening dose of capecitabine on Day-1.

On Cycle 1, Day 1, following an overnight fast of at least 8 hours, all patients in Cohort 2 are administered a single, morning dose of tesetaxel (27 mg/m²) orally, followed 2 hours later by capecitabine (825 mg/m²) within 10 minutes of a standard breakfast meal. PK sample collection occurs from pre-tesetaxel dose through 6 hours post-tesetaxel dose (i.e., 4 hours post-capecitabine dose). Patients take the evening dose of capecitabine (825 mg/m²) with a meal.

Subsequently, capecitabine (825 mg/m²) is administered orally twice daily (in the morning and evening after a meal, for a total daily dosage of 1,650 mg/m²) beginning with the morning dose on Day 2 through the evening dose on Day 14 of Cycle 1. Patients return to the clinic on Days 2, 7 and 14 of Cycle 1 following an overnight fast of 8 hours for administration of the morning dose of capecitabine within 10 minutes following a standard breakfast meal. For all other doses, patients self-administer capecitabine at home. PK sample collection on Days 2, 7, and 14 of Cycle 1 occurs from pre-capecitabine dose through 2 hours post-capecitabine dose.

Starting with Cycle 2, all patients in the second cohort are administered 27 mg/m² tesetaxel orally once every 21 days on Day 1 of each 21-day cycle, and 825 mg/m² capecitabine orally at twice-daily intervals beginning with the evening dose on Day 1 through the morning dose on Day 15 of each 21-day cycle.

The primary endpoint is objective response rate as adjudicated by an independent review committee. Secondary endpoints include duration of response as assessed by an independent review committee, progression-free survival as assessed by an independent review committee, disease control rate as assessed by an independent review committee and overall survival. Efficacy on CNS metastases is measured in patients with CNS metastases at baseline by CNS objective response rate and CNS duration of response as assessed by the CNS independent review committee.

### Example 5

### Clinical Study

Patients with CNS metastases secondary to breast cancer of any histology are recruited. Patients are treated with teseataxel monotherapy on Day 1 of each 21-day cycle. Treatment continues in 21-day cycles until the disease progresses, unacceptable toxicity is observed in the patient or other decision to discontinue treatment.

Efficacy on CNS metastases is measured by the CNS objective response rate and CNS duration of response.

### Example 6

### Clinical Study

Patients with CNS metastases secondary to breast cancer of any histology are recruited. Patients are treated with tesetaxel on Day 1 of a 21-day cycle and 14 daily doses of 1,650 mg/m² of capecitabine (825 mg/m² at twice-daily intervals) starting on Day 1 of the 21-day cycle for 14 consecutive 24-hour periods. Treatment continues in 21-day cycles until the disease progresses, unacceptable toxicity is observed in the patient or other decision to discontinue treatment.

Efficacy on CNS metastases is measured by the CNS objective response rate and CNS duration of response.

### Example 7

### Clinical Study

Patients with CNS metastases secondary to lung cancer of any histology are recruited. Patients are treated with tesetaxel monotherapy on Day 1 of each 21-day cycle. Treatment continues in 21-day cycles until the disease progresses, unacceptable toxicity is observed in the patient, or other decision to discontinue treatment.

Efficacy on CNS metastases is are measured by the CNS objective response rate and CNS duration of response.

### Example 8

### Clinical Study

Patients with CNS metastases secondary to lung cancer of any histology are recruited. Patients are treated with tesetaxel orally on Day 1 of each 21-day cycle, plus an inhibitor of PD-1 or PD-L1, such as nivolumab (360 mg) by 30-minute intravenous infusion on Day 1 of each 21-day cycle; pembrolizumab (200 mg) by 30-minute intravenous infusion on Day 1 of each 21-day cycle; or atezolizumab (1,200 mg) by 60-minute intravenous infusion (if first infusion is tolerated, all subsequent infusions may be delivered over 30 minutes) on Day 1 of each 21-day cycle. Treatment continues in 21-day cycles until the disease progresses, unacceptable toxicity is observed in the patient, or other decision to discontinue treatment.

Efficacy on CNS metastases is measured by the CNS objective response rate and CNS duration of response.

### Example 9

### Preclinical Study

The in vivo tissue distribution of tesetaxel was previously investigated in mice treated with ¹⁴C-tesetaxel at 4 mg/kg and sacrificed between 1-168 hours.¹ Two additional studies have been conducted in dogs and monkeys.

Methods:

Dogs and monkeys were dosed at 0.6 mg/kg and 1 mg/kg of ¹⁴C-tesetaxel, respectively, and tissue distribution was assessed at 336 hours post-dose (14 days). These doses equate to ˜44% of the 27 mg/m² dose in an ongoing Phase 3 clinical study. In vivo radioactivity concentrations in the cerebrum and cerebellum were compared to plasma concentrations and mean tumor GI₅₀ (concentration resulting in 50% tumor growth inhibition) determined by MTT assay in 23 tumor cell lines.

Results:

CNS penetration was observed following both oral (PO) or intravenous (IV) administration to the dogs and monkeys, and the CNS-to-plasma ratio was high for both the cerebrum and the cerebellum following both oral and IV administration (Table 1). Notably, Day 14 represents 4-6 half-lives after a single dose in dogs and monkeys following both routes of administration, suggesting the potential for even higher CNS-to-plasma ratios after multiple doses of tesetaxel. This suggests slower elimination from the CNS than from plasma. The absolute CNS concentrations of tesetaxel in dogs and monkeys at Day 14 exceeded the tumor GI₅₀ in 23 of 23 tumor cell lines, including 2 of 2 CNS tumor cell lines (Table 2). In a separate study, human plasma and CNS tissue binding were studied using rapid equilibrium dialysis and were shown to be equivalent at 98.2% and 98.9%, respectively.

TABLE 1
¹⁴C-Tesetaxel Dose and Exposure in Dog
and Monkey Tissues at 14 Days Post-dose

| | | | | Plasma | | |
|---|---|---|---|---|---|---|
| | Dose | | t_{1/2} | (ng | Cerebrum | Cerebellum |
| Species | (mg/kg) | Route | (days) | eq/mL) | (ng eq/g) | (ng eq/g) |
| Dog | 0.6 | PO | 4.0 | 0.9 ± 0.1 | 10.9 ± 4.0 | 6.1 ± 1.9 |
| | | IV | 3.0 | 0.5 ± 0.0 | 16.8 ± 2.9 | 7.8 ± 0.6 |
| Monkey | 1 | PO | 2.2 | 0.9 ± 0.2 | 6.5 ± 3.8 | 4.4 ± 1.7 |
| | | IV | 3.0 | 1.0 ± 0.0 | 21 ± 2.0 | 18 ± 1.0 |

TABLE 2
Tesetaxel CNS Levels Exceed Tumor GI₅₀^{a} in
Dogs and Monkeys Following Oral Administration

| | | ¹⁴C-Tesetaxel Radioactivity | | | |
|---|---|---|---|---|---|
| | | Concentration (ng eq./g or mL) | | | |
| | | 14 Days after Dosing | | | Cerebrum |
| | | Cerebrum | Plasma | Cerebrum/ | Concentration/ |
| | N | (Mean ± SD) | (Mean ± SD) | Plasma | Tumor GI₅₀ ^{b} |
| Dog ^{c} | 3 | 10.9 ± 4.0 | 0.9 ± 0.1 | 12x | 18x |
| Monkey ^{d} | 3 | 6.5 ± 3.8 | 0.9 ± 0.2 | 7x | 11x |

^{a}Concentration of drug required to inhibit growth by 50%
^{b} Mean GI₅₀ for tesetaxel across 23 tumor cells lines = 0.6 ng/mL
^{c} Single dose of 0.6 mg/kg (equivalent to 44% of a human dose of 27 mg/m²)
^{d} Single dose of 1 mg/kg (equivalent to 44% of a human dose of 27 mg/m²)

Discussion:

The CNS concentrations of tesetaxel in dogs and monkeys at Day 14 exceeded the tumor GI₅₀ in 23 of 23 tumor cell lines and 2 of 2 CNS tumor cell lines, suggesting that efficacious levels in the CNS are achievable (Table 2). The positive CNS-to-plasma ratio on Day 14 relative to the plasma pharmacokinetics and the dosing regimen in humans suggests that tesetaxel CNS levels may be even higher after multiple doses.

### REFERENCES

1. Ono et al, Biological and Pharmaceutical Bulletin 2004; 27(3):345-351.
2. Shionoya et al, Cancer Science 2003; 94(5):459-66.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

1. A method of treating a cancer in the central nervous system (CNS) of a human patient, comprising administering a therapeutically effective amount of tesetaxel systemically.

2. The method of claim 1, wherein the tesetaxel is administered orally.

3. The method of claim 1 or 2, wherein the cancer comprises a metastatic tumor.

4. The method of claim 3, wherein the metastatic tumor is a metastasis of a primary cancer selected from breast cancer.

5. The method of claim 4, wherein the breast cancer is hormone receptor positive.

6. The method of any one of claims 4-5, wherein the patient has previously received endocrine therapy.

7. The method of any one of claims 4-6, wherein the breast cancer is estrogen receptor positive.

8. The method of any one of claims 4-7, wherein the breast cancer is progesterone receptor positive.

9. The method of any one of claims 4-8, wherein the breast cancer is human epidermal growth factor receptor 2 (HER2) negative.

10. The method of any one of claims 4-9, wherein the breast cancer is hormone receptor positive and HER2-negative.

11. The method of any of claim 4, wherein the breast cancer is hormone receptor (HR) negative and HER2-negative.

12. The method of claim 3, wherein the metastatic tumor is a metastasis of a primary lung cancer, such as non-small cell lung cancer or small cell lung cancer.

13. The method of claim 1 or 2, wherein the cancer comprises a primary CNS tumor.

14. The method of claim 13, wherein the primary CNS tumor is an acoustic neuroma, astrocytoma, chordoma, CNS lymphoma, craniopharyngioma, glioma, medulloblastoma, meningioma, oligodendroglioma, pituitary tumor, primitive neuroectodermal or schwannoma.

15. The method of any one of the preceding claims, comprising administering the tesetaxel on day 1 of a 21-day cycle.

16. The method of any one of the preceding claims, further comprising administering a therapeutically effective amount of capecitabine.

17. The method of claim 16, comprising administering 14 daily doses of capecitabine starting on day 1 of the 21-day cycle.

18. The method of any one of claims 15-17, comprising repeating the 21-day cycle at least once.

19. The method of claim 18, comprising repeating the 21-day cycle until the cancer progresses or until unacceptable toxicity is observed.

20. The method of any one of claims 15-18, wherein administering a therapeutically effective amount of tesetaxel comprises administering 18-31 mg/m² of tesetaxel on day 1 of the 21-day cycle.

21. The method of any one of claims 15-20, wherein administering a therapeutically effective amount of tesetaxel comprises administering 27 mg/m² of tesetaxel on day 1 of the 21-day cycle.

22. The method of any one of the preceding claims, further comprising administering a therapeutically effective amount of an inhibitor of programmed cell death protein 1 (PD-1) or programmed death-ligand 1 (PD-L1), such as nivolumab, pembrolizumab, or atezolizumab.

23. The method of claim 22, wherein the inhibitor of PD-1 or PD-L1 is administered on day 1 of a 21-day cycle.

24. The method of claim 23, wherein the inhibitor is administered by intravenous infusion.

25. The method of claim 24, wherein the intravenous infusion occurs over 30 minutes.

26. The method of claim 24, wherein the intravenous infusion occurs over 60 minutes.

27. The method of any one of claims 16-26, wherein administering a therapeutically effective amount of capecitabine comprises administering 14 daily doses of capecitabine at twice-daily intervals.

28. The method of any one of claims 16-26, wherein administering a therapeutically effective amount of capecitabine comprises administering capecitabine in 28 doses at twice-daily intervals beginning on day 1 of the 21-day cycle.

29. The method of claim 28, wherein administering a therapeutically effective amount of capecitabine comprises administering a first dose of capecitabine on day 1 of the 21-day cycle and administering a final 28^{th} dose on day 15 of the 21-day cycle.

30. The method of any one of claims 16-29, wherein administering a therapeutically effective amount of capecitabine comprises administering 14 daily doses of 300-2,000 mg/m² of capecitabine beginning on day 1 of the 21-day cycle.

31. The method of any one of claims 16-30, wherein administering a therapeutically effective amount of capecitabine comprises administering 14 daily doses of 1,650 mg/m² of capecitabine beginning on day 1 of the 21-day cycle.

32. The method of claim 31, wherein administering a therapeutically effective amount of capecitabine comprises administering 825 mg/m² of capecitabine at twice-daily intervals for 14 consecutive 24-hour periods beginning on day 1 of the 21-day cycle.

33. The method of any one of claims 16-30, wherein administering a therapeutically effective amount of capecitabine comprises administering 14 daily doses of 1,750 mg/m² of capecitabine beginning on day 1 of the 21-day cycle.

34. The method of claim 33, wherein administering a therapeutically effective amount of capecitabine comprises administering 875 mg/m² of capecitabine at twice-daily intervals for 14 consecutive 24-hour periods beginning on day 1 of the 21-day cycle.

35. The method of any one of claims 16-30, wherein administering a therapeutically effective amount of capecitabine comprises administering 28 doses of 150-1,000 mg/m² capecitabine at twice-daily intervals.

36. The method of claim 35, wherein administering a therapeutically effective amount of capecitabine comprises administering 28 doses of 150-1,000 mg/m² of capecitabine at twice-daily intervals beginning with the first dose on day 1 of the 21-day cycle and ending with the 28^{th} dose on day 15 of the 21-day cycle.

37. The method of claim 35, wherein administering a therapeutically effective amount of capecitabine comprises administering 28 doses of 825 mg/m² capecitabine at twice-daily intervals.

38. The method of any one of claims 16-26, wherein administering a therapeutically effective amount of capecitabine comprises administering 28 doses of 825 mg/m² of capecitabine at twice-daily intervals beginning with the first dose on day 1 of the 21-day cycle and ending with the 28^{th} dose on day 15 of the 21-day cycle.

39. The method of claim 16-26, wherein administering a therapeutically effective amount of capecitabine comprises administering 28 doses of 875 mg/m² capecitabine at twice-daily intervals.

40. The method of claim 39, wherein administering a therapeutically effective amount of capecitabine comprises administering 28 doses of 875 mg/m² of capecitabine at twice-daily intervals beginning with the first dose on day 1 of the 21-day cycle and ending with the 28^{th} dose on day 15 of the 21-day cycle.

41. The method of any one of claims 1-40, wherein the patient has previously been treated with a taxane.

42. The method of any one of claims 1-40, wherein the patient has not previously been treated with a taxane.

43. The method of claim 41, wherein the patient has previously been treated with a taxane in the neoadjuvant or adjuvant setting.

44. The method of claim 41 or 43, wherein the taxane is paclitaxel, docetaxel or albumin-bound paclitaxel.
